(19) United States
(12) Patent Application Publication
(10) Pub. No.: US 2009/0270752 A1
(43) Pub. Date: Oct. 29, 2009

(54) Devices for Measuring Inspiratory Airflow

(76) Inventor: Robert E. Coifman, Millville, NJ (US)

Correspondence Address: Licata & Tyrrell P.C., 66 E. Main Street, Marlton, NJ, 08053, US

(21) Appl. No.: 12/497,021

(22) Filed: Jul. 2, 2009

Related U.S. Application Data
(63) Continuation of application No. 10/997,278, filed on Nov. 24, 2004.
(60) Provisional application No. 60/535,853, filed on Jan. 12, 2004.
(60) Provisional application No. 60/528,924, filed on Dec. 10, 2003.
(60) Provisional application No. 60/525,008, filed on Nov. 25, 2003.

(51) Int. Cl. A61B 5/087; A61M 16/10

(52) U.S. Cl. 600/538; 128/203.12; 128/203.15

(57) ABSTRACT

New applications of unidirectional airflow sensing in devices which measure inspiratory airflow and bidirectional airflow are provided. Such devices can be used for administration of a medication via inhalation, in spirometers and in devices which measure and monitor respiratory ventilation.

This patent application is a continuation of U.S. patent application Ser. No. 10/997,278, filed Nov. 24, 2004, which claims the benefit of priority from U.S. Provisional Patent Application Ser. No. 60/535,853, filed Jan. 12, 2004, U.S. Provisional Patent Application Ser. No. 60/528,924, filed Dec. 10, 2003 and U.S. Provisional Patent Application Ser. No. 60/525,008, filed Nov. 25, 2003, teachings of each of which are herein incorporated by reference in their entirety.

### INTRODUCTION

1. Field of the Invention

The present invention relates to new applications of stable, rugged and inexpensive unidirectional airflow sensing technologies described previously for measurement of human expiratory airflow, in devices which measure inspiratory airflow and bidirectional airflow, meaning measuring airflow during both inspiration and expiration. Exemplary devices of the present invention comprising an inspiratory airflow sensor include, but are in no way limited to, devices for administration of a medication or diagnostic challenge reagent via inhalation, devices measuring both inspiratory and expiratory airflow such as spirometers, and devices wherein monitoring and/or prompting of a subject to inhale air, specific gas mixtures, medicaments or diagnostic agents at programmed rates is required. Such inspiratory sensors are also useful in evaluating the performance of various inhalation devices.

2. Background of the Invention

Inexpensive mechanical peak flow meters (devices to measure peak expiratory flow rate, PEFR) have long been used to facilitate the day-to-day monitoring of asthma activity and to screen for otherwise unrecognized asthma or asthma exacerbations in such settings as schools. U.S. Pat. No. 6,447,459 describes an electronic flow sensor which very closely replicates the mechanical-to-electrical transducer function of the human ear, though with a different time constant and for a different purpose, as an array of levers and dampers similar to those of the human ear converts the displacement of a plate which is proportional to expiratory airflow into electrical output of a strain gauge. This flow measuring system serves as the sensor mechanism for the first electronic peak flow meter which is as rugged, stable and inexpensive as previously available mechanical peak flow meters. This device, described in U.S. Pat. No. 6,447,459, has an internal clock, calendar and timer and can electronically integrate flow rate over time to calculate one second forced expiratory volume (FEV1), generally accepted as a more sensitive parameter of changes in asthma activity than PEFR but not previously available for home recording because of a lack of stable, rugged and inexpensive devices with which to do so. The device of U.S. Pat. No. 6,447,459 can digitally record and download date and time-stamped measurements of PEFR and FEV1. Because of its ability to accurately measure flow rates as low as 30 ml/minute, this technology has the potential to record complete electronic expiratory flow patterns and calculate additional clinically useful pulmonary function parameters, such as 6-second forced expiratory volume, or FEV6.

The advantages of this inexpensive, rugged and stable flow measurement technology have not been extended to complete spirometers, because complete spirometers require the measurement of both inspiratory and expiratory airflow over the course of a single breath, and the mechanism of plates and levers that comprises the airflow sensor of this device can measure airflow in one direction, only.

The efficiency with which an inhaled medication is delivered to a specific target tissue within the respiratory tract depends on the size distribution of droplets or particles of medication in the aerosol being inhaled, the rate of inhalation of drug, and the timing and pattern of inhalation over the course of the respiratory cycle. For some inhaled medications with low cost, low bystander tissue toxicity and an immediate response to treatment, such as albuterol for use as a rescue medication for acute exacerbations of asthma, as long as significant amounts of drug get in, dosing efficiency and reproducibility are of little concern. Most long-acting or gradually acting inhaled asthma controller medications have a several-fold variation in indicated dosage range, depending on the response and inhalation technique of the individual patient. Patients using these medications usually achieve better outcomes if instructed in efficient inhalation techniques. Even with such teaching, however, the variation in fractional delivered dose between patients is often large. Significant variation in the fractional delivered dose is also common for individual patients over time, as unmonitored variations in inhalation technique and changes in asthma activity affect inspiratory breathing pattern.

Inhalation becomes a practical alternative to injection for a broad range of drugs that require repeated dosing and are not effectively and reproducibly absorbed by the oral route, if systemic drug delivery by the inhalation route can be made sufficiently reproducible and efficient. Advantages include avoiding the discomfort of injections, avoiding the risk of contaminated needle-stick injuries to caregivers, avoiding the need for lawful needle and syringe disposal, and in many cases, reducing the risks of infection and other adverse reactions to drug administration.

Numerous technologies have been developed to reproducibly generate aerosols of respirably-sized drug-containing particles. What is needed to make inhalation a preferred alternative to injection for the dozens of new genetically engineered disease-modifying drugs entering the marketplace is inexpensive, reliable, user-friendly technologies to achieve and coordinate optimal patterns of drug aerosol release and inhalation.

Computer-based systems have been developed to monitor and coordinate inspiration with drug aerosol release or generation. Some such systems, like the Akita inhaled drug delivery system marketed by Inamed GmbH (Germany), are “active”, prompting the patient who has been instructed in the use of the device, to breathe according to a pre-programmed or pre-defined pattern for an interval that includes the time of drug aerosolization. Others, like the Adaptive Aerosol Delivery system marketed by Profile Therapeutics, Inc. (UK), are “passive”: the system monitors the patient=s respiratory pattern and then releases drug aerosol at a point or during a time in the respiratory cycle that has been programmed to optimize drug delivery. The term “Smart Dosing” is used herein to refer to technologies that attempt to coordinate the generation of drug-containing aerosols with inhalation by the patient, to optimize efficiency and achieve a high degree of reproducibility of inhaled drug delivery. Active Smart Dosing technologies may employ auditory and/or visual prompts. Passive Smart Dosing technologies may coordinate the generation and release of drug-containing aerosols with the patient=s spontaneous respiratory cycle, and/or they may attempt to regulate the respiratory cycle, as well.

The term “Smart Dosing” as used herein is not meant to include devices that simply improve aerosolized drug availability but do not specifically coordinate aerosol generation with inhalation. By this standard, the valved holding chambers used to contain an aerosol of medication generated by a metered dose inhaler, for inhalation over a series of breaths by an infant or child too immature to inhale it effectively in a single breath, are not “Smart Dosing” devices.

Principles have been discovered according to which, for aerosols of certain particle sizes, inhalation at specified rates for different parts of the inspiratory cycle can result in more efficient and reproducible aerosol delivery to the lung, and, for systemically absorbed drugs, to the body. The feedback control of respiration required to reproducibly achieve these respiratory patterns is a primary application of what is termed herein “Smart Dosing.” The present invention meets the need for a rugged, reliable and inexpensive technology to achieve this goal.

Certain pulmonary diagnostic tests require that the subject breathe a specified gas at a specified or programmed rate. An example is measurement of change in pulmonary function following eucapni voluntary hyperventilation, a test with the potential to be a much safer way to evaluate exercise-associated shortness of breath in adults than exercise challenge tests, as it does not carry the risk of exercise challenge tests of provoking cardiac events if the shortness of breath turns out to be of cardiac origin. However, this test requires that subjects inhale a mixture of dry air with 5% added carbon dioxide (commercially available in tanks) at a rate equal to 85% of their calculated or estimated maximum voluntary ventilation rate for a period of six minutes. Presently available instruments are capable of measuring the total volume of gas inhaled during the 6 minute challenge, allowing retrospective calculation of how closely the subject's average ventilation rate approximates the target. There is presently no rugged, reliable and inexpensive device capable of displaying the subject's current ventilation rate in real time against a background showing the target level and an acceptable tolerance range, for use as a visual or auditory prompt. The availability of such a device would greatly increase the precision of this test, reduce the amount of training necessary for subjects to learn to maintain a satisfactory ventilation rate, and reduce the rate of test failure because of failure to maintain an acceptable ventilation rate in subjects who have difficulty mastering the technique without real time visual and/or auditory prompts.

### SUMMARY OF THE INVENTION

The present invention relates to new uses for stable, rugged and inexpensive unidirectional airflow sensing technologies in Smart Dosing devices which measure inspiratory airflow and bidirectional airflow.

The term “Smart Dosing” is used herein to refer to technologies that attempt to coordinate the generation of drug-containing aerosols with inhalation by the patient, to optimize efficiency and achieve a high degree of reproducibility of inhaled drug delivery. Active Smart Dosing technologies may employ auditory and/or visual prompts. Passive Smart Dosing technologies may coordinate the generation and release of drug-containing aerosols with the patient=s spontaneous respiratory cycle, and/or they may attempt to regulate the respiratory cycle, as well.

The term “Smart Dosing” as used herein is not meant to include devices that simply improve aerosolized drug availability but do not specifically coordinate aerosol generation with inhalation. By this standard, the valved holding chambers used to contain an aerosol of medication generated by a metered dose inhaler, for inhalation over a series of breaths by an infant or child too immature to inhale it effectively in a single breath, are not “Smart Dosing” devices.

Accordingly, one object of the present invention is to provide delivery devices equipped with inspiratory flow sensors for the inhalation of medications and diagnostic challenge reagents. Exemplary devices of this embodiment of the present invention include continuous aerosol generation devices such as a nebulizer fitted with a sensor of inspiratory flow rate, the output of which may be used for either active or passive feedback control of flow rate and to optimize the release of aerosolized medication for efficient and reproducible inhalation. Discrete puff dosing devices can also be fitted in accordance with the present invention with a sensor of inspiratory flow rate, the output of which may be used for either active or passive feedback control of flow rate and to optimize the release of aerosolized medication for efficient and reproducible inhalation.

In similar fashion, the present invention provides for incorporation of an inspiratory flow sensor into a device to measure and record the time course of inspiratory airflow through breath-powered dry powder inhalers.

Another object of the present invention is to provide an inexpensive spirometer which measures bidirectional airflow with one way flow sensors by incorporating two such sensors into the spirometer, positioned such that the placement and operation of each does not interfere with the accurate measurement of airflow through the other.

Yet another object of the present invention is to provide a simple, reliable and inexpensive device to measure and monitor a subject's rate of ventilation of a specified gas thereby facilitating the performance of tests and challenge procedures that require such ventilation at a specified or programmed rate for a specified period of time. Such devices are used to monitor and prompt subjects to inhale air or specific gas mixtures or medicaments or diagnostic agents at programmed rates that are needed for the accurate performance of various treatments, diagnostic tests and challenges and to evaluate the performance of various inhalation devices.

### BRIEF DESCRIPTION OF THE DRAWINGS

FIGS. 1A and 1B shows a side view of a Smart Dosing module mounted in a cap for one of the presently marketed microporous membrane nebulizers. The Smart Dosing module of FIG. 1A fits over the nebulizer mechanism of FIG. 1B. The solid arrow designates the path of inspired air while the dashed arrow designates the flow of exhaled air through an escape valve.

FIG. 1C shows an embodiment of a display unit for use with the Smart Dosing module of FIG. 1A.

FIG. 2 provides a diagram of a back view of the Smart Dosing module of FIG. 1 equipped with an inspiratory flow sensor designed to fit a presently marketed microporous membrane nebulizer. Arrows depict the path of inspired air around the drug reservoir of the nebulizer.

FIG. 3 provides a diagram depicting positioning of separate inspiratory and expiratory flow sensors to minimize interference with each other's operation in a spirometer. The dashed arrows designate the path of inspiratory air flow and the solid arrows designate the path of expiratory air-flow.

FIG. 4 is a diagram of an embodiment of an inspiratory flow sensor for use in tests for which the subject must maintain a target rate of ventilation of a specified gas. The device may be fitted to non-rebreathing systems in which exhaled air (designated by (E)) is vented to the outside or to devices that measure various components, or to rebreathing systems in which exhaled air may pass through various devices to measure and/or remove various components before being returned to the inspiratory reservoir.

FIG. 5 is a diagram showing placement of an inspiratory flow sensor on a breath-powdered dry powder inhaler. Arrows depict the flow path of ambient air.

### DETAILED DESCRIPTION OF THE INVENTION

A number of physical and physiological principles have been discovered that govern the deposition in various tissues of the airway of inhaled drug aerosols. Many of these depend on the particle size distribution of the aerosol and on the user achieving specified inspiratory airflow patterns which minimize particle deposition on parts of the airway more proximal than the intended target tissues, result in delivery of the particle to the parts of the airway in which one wants them to settle out, and maintain the particle there long enough for maximal, reproducible target tissue delivery. Within the range of particle sizes for which appropriately programmed flow patterns can achieve effective target tissue delivery, larger particles are generally easier to generate, and more efficient carriers of drug because volume (and thus drug carrying capacity) varies as the cube of particle diameter. However, large particles have a much greater tendency than smaller ones to leave the air stream and impact on the walls of whatever they are flowing through (whether it be part of a device or of the airway) under conditions of turbulent airflow. Increasing the speed of airflow through any size opening increases turbulence.

For purposes of delivery of a drug or diagnostic challenge reagent by inhalation, the airway begins at the mouth, reaches its narrowest point in the throat, and divides into large numbers of small branches as it progresses into the lungs. As it does this, its total cross-sectional area increases rapidly and dramatically. At a level at which the total cross-sectional area of the airway is 100 times the cross-sectional area at the narrowest part of the throat, air will be moving only 1% as fast as air at the throat as the user continues to inhale. At the level at which total cross-sectional area has increased to 1000 times that of the cross-sectional area of the throat, air speed has slowed to 1/1000 of its velocity in the throat. Thus, even if one is inhaling as fast as one can, an individual mouthful of air does not have to move very far into the lung to reach a level at which the airway has branched sufficiently, and cross-sectional area has increased sufficiently, so that this mouthful of air is moving very slowly, with essentially no turbulence, even if the user is inhaling at maximum speed. A. R. Clark et al reported at the June 2003 Congress of the International Society for Aerosols in Medicine, that a mouthful-sized bolus of easy-to-generate “large” or “coarse” droplets can be delivered to the distal airway with reproducible high efficiency if it is inhaled very slowly, without generating turbulence and settling out by impaction, for enough time to pass into the part of the airway in which cross-section is large and flow rate is slow under any condition of respiration. The user can then inhale to fill his or her lungs to capacity as rapidly as possible, carrying the mouthful of air containing the medication to the periphery, where it settles out in tissues from which it can be efficiently absorbed, while the user holds his or her breath. These studies were performed with a laboratory-grade flow sensor connected to a computer displaying prompts for the user on its screen.

In the present invention devices are provided which perform the same or similar functions using an inexpensive, rugged and reliable Smart Dosing module comprising a flow sensing mechanism such as described in U.S. Pat. No. 6,447,459, herein incorporated by reference in its entirety, or any other comparably stable, rugged and inexpensive airflow sensor to measure inspiratory airflow. The term “Smart Dosing” is used herein to refer to technologies that attempt to coordinate the generation of drug-containing aerosols with inhalation by the patient, to optimize efficiency and achieve a high degree of reproducibility of inhaled drug delivery. Active Smart Dosing technologies may employ auditory and/or visual prompts. Passive Smart Dosing technologies may coordinate the generation and release of drug-containing aerosols with the patient=s spontaneous respiratory cycle, and/or they may attempt to regulate the respiratory cycle, as well.

The term “Smart Dosing” as used herein is not meant to include devices that simply improve aerosolized drug availability but do not specifically coordinate aerosol generation with inhalation. By this standard, the valved holding chambers used to contain an aerosol of medication generated by a metered dose inhaler, for inhalation over a series of breaths by an infant or child too immature to inhale it effectively in a single breath, are not “Smart Dosing” devices.

In accordance with the present invention, these inspiratory airflow sensors or flow meters can be incorporated into any device which measures inspiratory airflow or bidirectional airflow, meaning measuring airflow during both inspiration and expiration. Such devices of the present invention comprising an inspiratory airflow sensor include, but are in no way limited to, devices for administration of a medication or diagnostic challenge reagent via inhalation, devices measuring both inspiratory and expiratory airflow such as spirometers, and devices wherein monitoring and/or prompting of a subject to inhale air, specific gas mixtures, medicaments or diagnostic agents at programmed rates is required. Such inspiratory sensors are also useful in evaluating the performance of various inhalation devices.

In one embodiment of the present invention, the Smart Dosing module is coupled to or incorporated into a medical aerosol-generating device. This use of a sensor such as described in U.S. Pat. No. 6,447,459 is outside the scope of that patent, which only teaches use for measurement of exhaled or expired air. In the present invention, the sensor may be used to confer either active or passive “Smart Dosing” capability on either discrete puff or continuous aerosol generating devices.

In all medical aerosol generating devices, a medication-containing aerosol is released into a space, referred to herein as the aerosol holding area, from which it is inhaled. In the present invention, a one way flow sensor such as described in U.S. Pat. No. 6,447,459 or an alternative, comparably rugged, stable and inexpensive flow sensor with comparable accuracy across the human respiratory flow range and similar electronic outputs, is placed in the intake channel with the minimum volume of dead space between the sensor and the aerosol generation device, consistent with the design and cleaning requirements of the device. Depending on the material to be aerosolized and the cleaning requirements it creates for the device, it may be necessary to position a low resistance one way flap valve between the flow sensor and the aerosol holding area. Special cleaning requirements for some uses may mandate that the sensor and its accompanying electronic elements be separable from the aerosol holding area.

As in the expiratory flow measuring application described in U.S. Pat. No. 6,447,459, a sensor module used in the presence invention comprises a microprocessor with the ability to record flow, time, and, by integrating flow over time, volume of air inhaled through the device after a signaling event. In some embodiments, the microprocessor records these parameters for a series of “test” breaths prior to inhalation. In some embodiments to be used with medication aerosols which are inhaled over the course of multiple breaths from continuous aerosol generating devices, the microprocessor component of the sensor records the inspiratory flow pattern for each breath, and uses the measurements from a specified number of preceding breaths to compute the dosing time and target inhalation pattern for the next breath. In some embodiments the sensor module is capable of accepting external data and/or storing and/or exporting recorded flow measurement data. In this embodiment, data storage, power if required, and an input/output capacity must be provided.

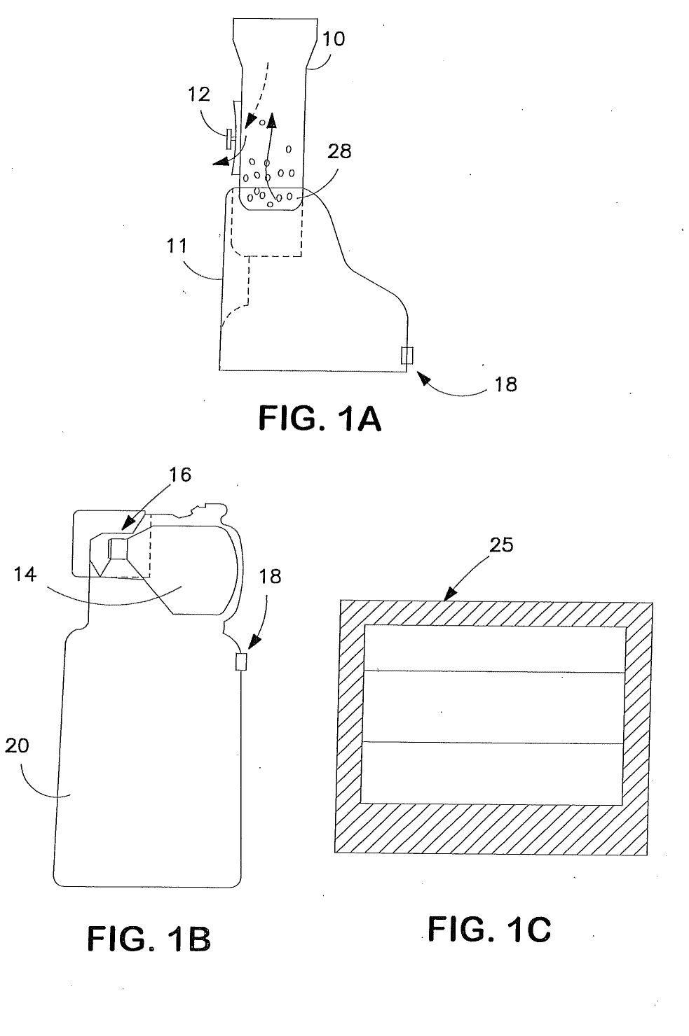

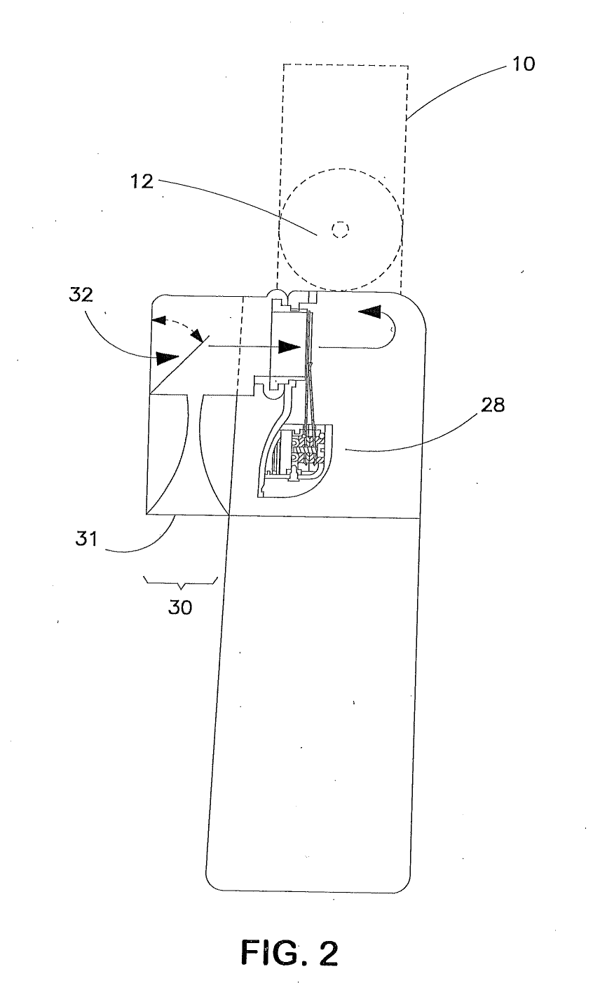

The physical design of the sensor module's shell will vary according to the physical design of the aerosol generating device with which it is designed to be used. An exemplary embodiment designed to fit a presently marketed microporous membrane nebulizer is shown in FIGS. 1 and 2. In the embodiment depicted in FIGS. 1 and 2, the sensor module shell forms a cap 11 which fits over the drug reservoir 14 and membrane of the nebulizer mechanism 20. The cap 11 fits so that contacts 18 on the nebulizer mechanism 20 and the cap 11 connect to provide power to the sensor module 28 via the on/off switch of the nebulizer.

To facilitate manufacture, servicing, and to be compatible with disposable discrete puff aerosol generating devices, it is generally preferred that the sensor module be constructed as a separate physical device that plugs in or otherwise attaches to the aerosol generating device, with a sufficiently tight seal to prevent entry of extraneous air into the aerosol holding area and with necessary electrical contacts for power and communication between the flow sensor module and the aerosol generating device.

For active Smart Dosing, in which the output of the sensor module must provide a visual or auditory prompt to enable the user to maintain a programmed inspiratory cycle and timed breath-holding, the sensor module must have appropriate outputs for a display module 25 (see FIG. 1C). For passive Smart Dosing, in which the output of the sensor module microprocessor directly controls the air supply available to the user, the intake channel through which air flows from the environment to the sensor will have two branches, one that has a flow-limiting inlet 31 to limit the user=s rate of inhalation without need for a visual or auditory prompt, and the other that is closed by a vane or valve 32 until the microprocessor determines that the bolus of inhaled drug has passed into the low flow region of the airway, at which time the vane or valve 32 is open so that the user can then inhale rapidly to maximum inspiratory capacity. The vane or valve may either be incorporated into the sensor module or else be constructed as a separate, flow-regulating module 30 such as shown in FIG. 2.

Some embodiments of the aerosol generating devices used with the present invention may have a one way flow valve or valves in the mouthpiece and/or mask of the device. As shown in FIG. 2, for these embodiments the mouthpiece 10 can be elongated as compared to the currently marketed device by approximately 0.75 inches to accommodate an escape valve 12 for expired air. Use of an escape valve 12 is optional for active dosing systems since many users can learn to remove the mouthpiece from their mouth while exhaling. For some users having difficulty responding to the inspiratory flow prompts of active Smart Dosing when they also have to remember to remove the device from their mouth to exhale and put it back before the next breath; however a mouthpiece that has an escape valve may be preferred. Depending on the patient being treated, passive Smart Dosing systems may use a mask as an alternative to a mouthpiece. An escape valve for expired air can be incorporated into a mask, as well.

A second one-way valve, to prevent backflow of exhaled respiratory secretions into the aerosol generation device, may be incorporated into the mouthpiece or placed between the aerosol generation device and the mouthpiece, for uses in which it is desirable that multiple users or patients be able to inhale from the same aerosol generation device without need for disinfection of the complete device between users. Such valves must be of much higher precision and reliability than those needed to vent expired air: to be acceptable for this use they must completely and reliably prevent the backflow of exhaled respiratory secretions from one user into any portion of the aerosol generation device from which infectious contents could infect other users.

Any obstacle in the channel between the source of the medication aerosol and the airway, including a second one-way valve, will create turbulence during inhalation, resulting in impaction and loss of drug, the extent of which will depend on valve design. There may be clinical applications of this technology for which this loss of drug is an acceptable price to pay (and for which drug dose can be increased to compensate) for the convenience of being able to treat multiple patients in sequence with the same nebulizer without risk of cross-infection. Such drug loss may be too variable to be acceptable for other applications, however.

For most embodiments, the onset of inspiratory airflow will be the signaling event that either triggers a discrete puff device to release a puff containing a unit dose of medication, or turns on a continuous aerosol generation device. Depending on the algorithm built into the microprocessor, a continuous aerosol generation device will be turned off following a programmed interval of time after it is turned on or after a programmed or calculated volume of air has passed through the sensor. The user is prompted to continue to inhale at a constant, very slow flow rate in active embodiments, or the volume of air available for inhalation continues to be restricted to maintain this rate, in passive embodiments, to allow the bolus of inhaled drug to move without turbulent airflow and impaction, far enough into the lungs that it will continue to move slowly, without turbulence and impaction, even if inspiratory flow rate is then increased.

A. R. Clark et al demonstrated in the above-referenced study that for aerosols with a mass median aerosol diameter of 6.5 microns, 120 ml/minute is an effective inhalation rate for the slow, steady inhalation phase of the drug inhalation cycle. Further, it is believed that tolerance limits of +/−10 to 20 ml/minutes will not adversely affect the efficiency or reproducibility of drug delivery. However, different inhalation rates and tolerance limits may apply to aerosols of different sizes or to drugs targeted to different levels of the airway, there being data suggesting that different classes of topically acting asthma medications may be more effective if targeted to bronchi of different diameters, the larger bronchi being located more proximally in the airway and the smaller ones more distally. Processors can be made for which the various prompt and display parameters are programmable, so that specific flow targets and tolerances need not be hard-wired into the device.

The sensor module of the present invention has electrical connections to trigger aerosol release from the discrete puff device and turn a continuous flow aerosol generation device on and off. Thus, the aerosol generation devices must have actuators (for discrete puff devices) and switches (for continuous flow devices) that can be actuated and turned on and off by signals from the sensor module. Sensor modules designed for active Smart Dosing for use in the present invention are also connected to a display module. In one preferred embodiment, the sensor module is connected to the display module by means of a 4 foot cable that is not hard wired into either module, so it can easily be unplugged and replaced if it wears out. Sensor modules designed for passive Smart Dosing also have contacts to communicate with the flow regulating modules to which they are connected.

A visual display module such as depicted in FIG. 1C for use in the present invention preferably comprises a screen able to display the user's current and recent inspiratory flow rates against a background displaying the target range. A preferred embodiment will use the same display format commonly used for electrocardiographic tracings. The graph of past measurements together with its time scale moves across the screen to the left as new data points are displayed near the center. The vertical axis of the graph, at which new data is displayed as older data moves to the left, will be at or slightly to the right of the center instead of at the right edge of the screen, so that programmed or calculated changes in inhalation activity (going from slow to fast inhalation when the bolus of aerosolized drug is calculated to have entered the low flow portion of the airway, beginning to hold breath, ending breath-holding) are visible to the right of the axis for several seconds before they have to be implemented, and move leftward to intersect the vertical axis at the moment that the patient should implement them. For both discrete puff devices and continuous aerosol generation devices, the user will be prompted to continue to inhale at a slow, steady rate for a time interval following either release of the puff or cessation of aerosol generation, with the length of this time interval calculated to allow the bolus of aerosolized drug to move far enough into the lung to reach an area of permanent low flow rate and linear airflow. The length of this time interval may be pre-programmed, it may be the time to inhalation of a pre-programmed volume of air following cessation of aerosol generation, or it may be a time determined by the operation of any other algorithm found to be effective for the facilitation of maximal, reproducible drug delivery.

At the end of the programmed or calculated interval for steady, slow inhalation, the display will prompt the user to, for example, “Breathe In Fast.” When the rate of inspiratory airflow decreases to zero, at maximal inhalation, the prompt on the screen will change, for example, to, “Hold Breath,” and the time line will indicate the amount of time remaining that the user should hold his or her breath to achieve maximal and reproducible drug deposition.

Some embodiments of the display module may contain auditory prompts, to alert the user before and/or at the key transition points of the change from slow, steady inspiration to maximal inspiration to total lung capacity, and again when the user can stop holding his or her breath. Some embodiments may be designed to offer a complete set of auditory prompts as an alternative to visual prompts, for users who are visually handicapped. Preferred embodiments of totally auditory prompts may employ some or all of different sounds, pitches, tones, volumes and intervals between tones, beats or beeps, with or without electronic playback of segments of specific musical compositions, with optional earphone use to avoid disturbing or distracting others in the area. A preferred embodiment of an auditory prompt for slow breath-holding will involve a steady, mid-range tone when the user is inhaling at the target rate, addition of a second, pulsed tone of a higher pitch with increasingly frequent beats when the user is inhaling at faster than target rate but within the tolerance limit, and upward-moving arpegios of increasing range as the user inhales with increasing speed above the tolerance limit. The same embodiment would use a pulsed tone of lower frequency than the steady baseline tone for slower than target inhalation rate within the tolerance limit, and increasingly long downward-moving arpegios for inhalation at rates progressively less than the lower tolerance limit. The user's or programmer's choice of any of a group of several second-long common musical sequences with predictable endpoints, played so as to be easily distinguishable from the above-described inspiratory flow rate prompts, could be used to prepare the user for the switch points of transition from slow steady inhalation to maximal, fast inhalation and end of breath-holding, and other sound patterns or musical excerpts could be used as prompts during maximal inspiration and breath-holding.

Preferably, both visual and auditory display modules contain their own source of power, which will generally be alkaline AA batteries, possibly with optional use of AC adapters.

When the sensor is to be used with an electrically powered aerosol generating device such as a microporous membrane nebulizer, it will generally be most expedient for the sensor to draw power for all functions except memory from the aerosol generating device. When used with discrete puff aerosol generating devices that have no electrical power requirement other than to enable the flow sensor to trigger actuation, it may be more practical to design space for one or two AA or AAA batteries in the sensor module shell and let this be the power source for the switch that releases what for most discrete puff devices will be a cocked spring-powered actuation mechanism. The most practical power source for flow regulatory modules of passive Smart Dosing devices will depend on the type and other design features of each device.

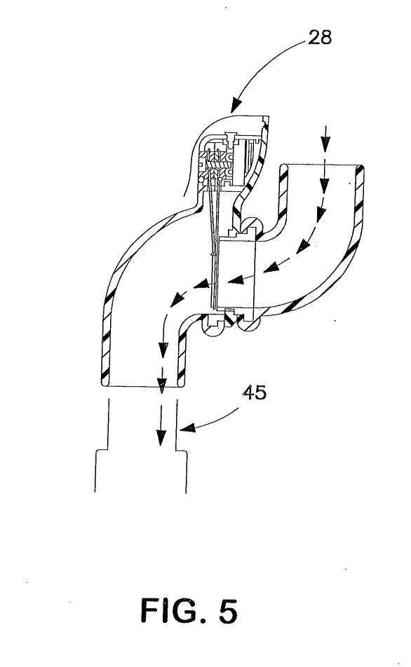

Inspiratory flow sensors or meters can also be incorporated into breath-powered dry powder inhalers in accordance with the present invention. A exemplary breath-powered powder inhaler 45 fitted with an inspiratory flow sensor or meter 28 in accordance with the present invention is depicted in FIG. 5.

Breath-powered dry powder inhalers of various design are simple and effective devices for the delivery of various medications and bronchoprovocation challenge reagents to the respiratory tract. Different devices in this class require inspiratory flow rates of 15 to 120 liters per minute to generate enough turbulence in the device to aerosolize the powdered medication or medication/carrier mix. These rates are 125 to 1000 times the 120 ml/minute target rate of inhalation to optimize small airway delivery for aerosols generated by other means, in a previously described embodiment. As a result of the higher inspiratory flow rate there is greater turbulence in the mouth, the throat and the proximal airway (before the airway branches sufficiently to reach the cross-sectional area beyond which flow is always slow), resulting in greater drug impaction and deposition in these sites. Nonetheless, for many drug/device combinations, effective target tissue delivery with dry powder inhalers compares favorably with or even exceeds that achievable with presently available pressurized metered dose inhalers. At this time, however, there is no simple, reliable and widely applicable technology for the measurement of inspiratory flow rate through devices of this type. Incorporation of an inspiratory flow meter into a breath-powered dry powder inhaler in accordance with the present invention will facilitate patient training, allow for real-time prompting, and facilitate the selection of appropriate inhalation devices for individual patients when devices with different resistance to inspiratory airflow are available. Further, the ability to measure inspiratory flow will facilitate the design of improved breath-powered dry powder inhalers.

The same considerations apply to inhalation of diagnostic challenge reagents delivered via breath-powered dry powder inhalers. Patients will perform these tests with less confounding of results by poor or variable technique if they can be trained to inhale at target flow rates and if they perform these tests with properly selected inhalers. These devices can be used to identify deviations from prescribed inhalation pattern, which may explain unexpected results for which this could be a cause. The availability of inspiratory flow meters for these devices will facilitate the design of improved devices for diagnostic inhalation challenge protocols using breath-powered dry powder inhalers, as well as for therapeutic drug delivery from devices of this type.

Inhalation from a breath-powered dry powder inhaler usually takes about one second, a time interval too short for the type of visual prompts described herein for use with continuous aerosol generation and non-breath-powered discrete puff aerosol generation inhaled drug delivery systems. Recording dry powder inhaler inspiratory flow-volume tracings for coaching and training, however, offers a way to improve patient performance for more effective and reproducible delivery of therapeutic drugs and greater standardization in inhaled drug diagnostic challenge procedures. When the same drug can be administered with dry powder inhalers which differ in intrinsic resistance, recording of inspiratory flow tracings from several empty devices (without medications) may facilitate the choice of the most appropriate device for each individual patient. Recording of inspiratory flow-volume curves during diagnostic inhalation challenge procedures will provide a previously unavailable confirmation that each challenge dose was inhaled effectively, and permit immediate identification of challenges in which the drug has not been inhaled as prescribed. The ability to study the effect of changes in various dry powder inhaler design parameters on the inspiratory flow-volume patterns of patients with different diseases and different levels of pulmonary function will facilitate the design of better dry powder inhalers. For these drug delivery devices simpler auditory prompts similar to those discussed herein for some classes of nebulizers are expected to improve patient performance with dry powder inhalers, as well.

Most present day dry powder inhalers do not have a standard air intake conduit but simply allow outside air to enter through one or more intake vents placed in the simplest way compatible with the operation of the device. It may be necessary to redesign some of these devices to either fit a “standard” dry powder inhaler inspiratory flow sensor, or to fit an adapter that enables these devices to be used with a “standard” inspiratory flow sensor.

As will be understood by those skilled in the art upon reading this disclosure, nebulizers, discrete puff dosing devices and breath-powered powder devices merely serve as three examples of drug or diagnostic reagent delivery devices into which an inspiratory flow sensor or meter can be incorporated. The present invention is not meant to be limited to these three types of dosing devices but rather to the broader aspect of incorporation of a rugged, reliable and inexpensive inspiratory flow meter into any inhalation delivery device.

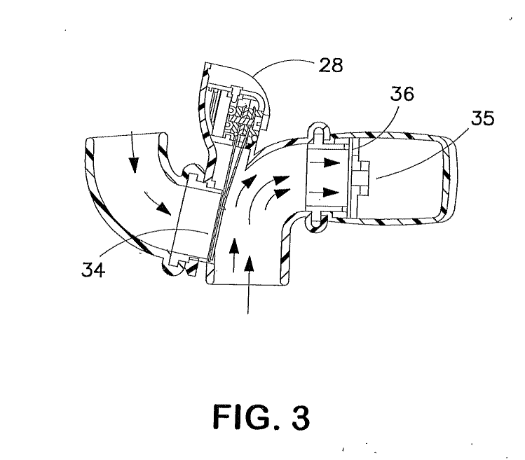

The airflow sensor described in U.S. Pat. No. 6,447,459 or a comparable alternative airflow sensor also provides a useful sensor for a spirometer. Further, its low cost and ability to record data for Internet transmission renders such a spirometer practical for home use. Presently available airflow sensing technologies that allow bidirectional flow rate measurement are less stable, less rugged and/or considerably more expensive than the flow measuring technology described in U.S. Pat. No. 6,447,459. Accordingly, home use is limited. Spirometry requires the measurement and recording of both inspiratory and expiratory airflow. The novel concept that allows the construction of a full-function spirometer with stable, rugged and economical one way airflow sensing devices is to incorporate two such sensors in the same device, one to measure expiratory airflow and the other to measure inspiratory airflow, with each positioned in the device in such a way that it does not mechanically interfere with the other and does not create enough turbulence to interfere with accurate flow measurement by the other. As these devices are not used to deliver aerosolized medications, droplet impaction is not a problem as it is in devices that deliver medications for inhalation. A preferred spirometer of the present invention uses paired sensors of the type described in U.S. Pat. No. 6,447,459. Alternative embodiments are to use paired unidirectional flow sensors of other types which share the features of ruggedness, stability, compatibility with electronic recording and transmission of data, simplicity of use and maintenance, and low cost. A spirometer depicting one exemplary embodiment of the positioning of two sensors, an inspiratory flow sensor 28 and an expiratory flow sensor 35, each equipped with a flow sensor plate 34 and 36 so that one does not interfere with operation of the other, is shown in FIG. 3. As will be understood by the skilled artisan upon reading this disclosure however, alternate positioning can be used.

The airflow sensor described in U.S. Pat. No. 6,447,459 or a comparable alternative airflow sensor also provides a useful sensor for a simple, reliable and inexpensive device to measure and monitor a subject's rate of ventilation of a specified gas, to facilitate the performance of tests and challenge procedures that require such ventilation at a specified or programmed rate for a specified period of time. In one such test, representative of this class of uses, the subject must breathe in and out for six minutes at a rate calculated to be 85% of his or her estimated one minute maximum ventilatory capacity, inhaling a specified gas mixture that is released into a reservoir which is usually a large, heavy duty balloon, either via continuous flow for a non-rebreathing system, or, if the gas is simply dry room air, optionally as a single fill of a larger balloon from which water vapor and carbon dioxide are removed prior to return. This is called a re-breathing system. Such systems contain valves to properly direct inhaled and exhaled gas and to prevent excessive pressure build-up in the system.

Placement in the inspiratory flow channel of such a system of a flow sensor of the type described in U.S. Pat. No. 6,447,459 or a comparable alternative airflow sensor, will allow real time measurement of the subject's ventilation rate and permit it to be displayed against a background of the target ventilation rate and programmed tolerances as a visual prompt, translated into sounds for an auditory prompt, or displayed as a visual prompt with auditory enhancements, in manners similar to those described as alternative display embodiments for active feedback monitoring of inspiratory flow rate for Smart Dosing of aerosolized medications by inhalation.

There are two differences between these two applications, however. The first is that for feedback monitoring of ventilatory rate, subjects will be inhaling and exhaling air at relatively high flow rates. This does not create a problem for the strain gauge-based flow sensor described in U.S. Pat. No. 6,447,459, as it is capable of accurate measurement across the full range of human respiratory airflow. To meet the requirement of comparability, alternative sensors must be comparably accurate over the same range. The second difference is that for this use, the parameter needing display is not real time momentary airflow through the sensor module, but the current average rate at which gas is being inhaled from the reservoir. In this use, the electronic component to which the sensor is connected is designed to integrate momentary inspiratory airflow over time span of multiple breaths to obtain a record of inhaled volume, and divide this number by the time over which the integration has taken place.

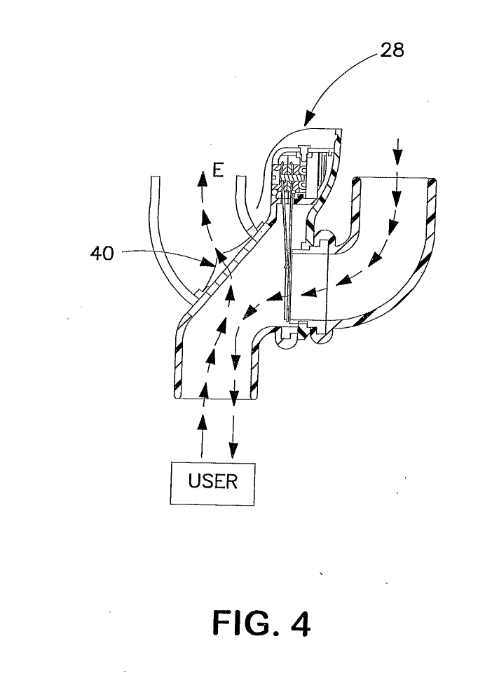

A sensor module constructed for this use need not have its own valves if it is placed in a part of the inspiratory gas flow circuit upstream from the valve(s) separating the flow paths of inspired and expired air going to and from the subject. For systems in which this is not practical, sensors may be mounted in assemblies equipped with valves, such as the one way exhaust valve 40 as illustrated in FIG. 4.

In an alternative embodiment, the same sensor is turned around and placed in the outflow path and measures the flow of exhaled rather than inhaled air. The electronics and display functions are the same whether the sensor is positioned to measure inspiratory or expiratory flow. The difference is that when the device is positioned to measure inspiratory airflow, the subject makes his or her ventilation level go up on the display by sucking in more air more quickly, while when it is positioned to measure expiratory airflow, the subject makes the value on the display panel go up by blowing out harder and faster.

Various display parameters may be effective as prompts. One embodiment which a priori seems to be a practical choice would display both the average rate of ventilation since the start of the test (total volume of gas inhaled since start of test divided by time since start of test) and the current rate of ventilation (volume of gas inhaled for most recent 3-10 seconds divided by that interval of time) on a graph display moving from right to left. Both ventilation values (average since start of test and current) will be displayed against a background showing target and tolerance limits, with the subject instructed to try to keep the current rate at a value within the displayed tolerance limits that keeps the average ventilation rate for the test as close as possible to the target value.

Superiority of inspiratory vs. expiratory airflow measurement for the ventilation meter/monitor may vary. It may be that some subjects deliver better test performance with the sensor positioned to measure inspiratory airflow and others deliver better performance with measurement of expiratory airflow, in which case it will be most pragmatic to make it with fittings that enable a single device to be configured in either position.

1. A device for administration of a medication via inhalation comprising:
a holding area from which said medication is inhalable;
an intake channel for delivering air to said holding area;
a sensor module comprising a sensor of inspiratory flow rate;
a microprocessor connected to said sensor for recording inspiratory flow rate measured by the sensor and time for providing active feedback control via a visual or auditory prompt which enables a user to maintain a programmed inspiratory cycle and timed breath-holding or for providing passive feedback control via an air supply control means for controlling the air supply available through said intake channel;
characterized in that said sensor comprises a plate moveable between a first position for blocking the intake channel and a second position for permitting inspiratory airflow through the intake channel to the holding area so that said device accurately measures an effective inhalation rate slow enough to prevent turbulence in a proximal airway of the lung based on the displacement of the plate of the sensor between the first and second positions while then permitting a user to inhale to fill his or her lungs to capacity as rapidly as possible, thereby carrying the bolus of air containing the medication to the lung periphery, where the medication settles out in tissues from which it can be efficiently absorbed, while the user holds his or her breath.

2. A device according to claim 1, wherein the medication is administered via continuous aerosol generation device comprising a nebulizer fitted with the sensor module comprising the sensor of inspiratory flow rate and the microprocessor, output of which is used for active or passive feedback control of flow rate and to optimize release of aerosolized medication for efficient and reproducible inhalation.

3. A device according to claim 1, wherein the medication is administered via a discrete puff dosing device fitted with the sensor module comprising the sensor of inspiratory flow rate and the microprocessor, output of which is used for active or passive feedback control of flow rate and to optimize release of aerosolized medication for efficient and reproducible inhalation.

4. A device according to claim 1, wherein the medication is administered via dry powder inhaler fitted with the sensor module comprising the sensor of inspiratory flow rate connected to the microprocessor which records inspiratory flow rate measured by the sensor and time so that the sensor module provides active or passive feedback control.

5. A device according to claim 1, wherein the sensor module further comprises a damper for damping movement of the plate.

6. A device according to claim 1, wherein the sensor module further comprises a strain gauge for measuring the displacement of the plate for determining the inspiratory flow rate.

7. A device for measuring bidirectional respiratory airflow comprising:
an inspiratory flow channel;
an inspiratory one way flow sensor with a plate positioned in the inspiratory flow channel to move between a first position for blocking the inspiratory flow channel from exhaled air and a second position for permitting inspiratory airflow through the inspiratory flow channel so that the inspiratory flow sensor is able to determine the inspiratory flow rate based on the displacement of the plate between the first and second position;
an expiratory flow channel; and
an expiratory one way flow sensor with a plate positioned in the expiratory flow channel to move between a first position for blocking the expiratory flow channel from inhaled air and a second position for permitting expiratory airflow through the expiratory flow channel so that the expiratory flow sensor is able to determine expiratory flow rate based on the displacement of the plate between the first and second position;
wherein placement and operation of the inspiratory one way flow sensor does not interfere with the accurate measurement of airflow through the expiratory one way flow sensor and placement and operation of the expiratory one way flow sensor does not interfere with the accurate measurement of airflow through the inspiratory one way flow sensor.

8. A device for measuring and monitoring ventilation in real time, comprising:
an inspiratory rebreathing flow channel of a non-rebreathing or a rebreathing respiratory ventilation system;
a sensor module comprising a sensor of inspiratory flow rate positioned in said inspiratory rebreathing flow channel;
a microprocessor connected to said sensor for recording inspiratory flow rate measured by the sensor and time for providing active feedback control via a visual or auditory prompt which enables a user to maintain a programmed inspiratory cycle and timed breath-holding or for providing passive feedback control via an air supply control means for controlling the air supply available to a user;
characterized in that said sensor comprises a plate moveable between a first position for blocking the inspiratory rebreathing flow channel and a second position for permitting inspiratory airflow through the inspiratory rebreathing flow channel, wherein the sensor module is able to determine the inspiratory flow rate based on the displacement of the plate between the first to second positions.
